(19) United States
(12) Patent Application Publication
(10) Pub. No.: US 2026/0232252 A1
(43) Pub. Date: Aug. 13, 2026

(54) METHOD OF DETECTING NEURAL SIGNALS, DETECTION DEVICE, AND NEURAL SIGNAL MEASURING SYSTEM

(71) Applicant: SAMSUNG ELECTRONICS CO., LTD., Suwon-si (KR)

(72) Inventors: Takhyung LEE, Suwon-si (KR); Joonseong Kang, Suwon-si (KR); Kitae Park, Suwon-si (KR); Hyeokki Hong, Suwon-si (KR)

(73) Assignee: SAMSUNG ELECTRONICS CO., LTD., Suwon-si (KR)

(21) Appl. No.: 19/279,578

(22) Filed: Jul. 24, 2025

(30) Foreign Application Priority Data
Feb. 12, 2025 (KR) 10--2025-0018290

(51) Int. Cl. A61B 5/372; A61B 5/00

(52) U.S. Cl. CPC A61B 5/372; A61B 5/7246; A61B 5/7264

(57) ABSTRACT

There is provided a method of detecting neural signals, a detection device, and a neural signal measuring system including dividing a neural signal measured via multiple channels into signal sections of a specified length, determining whether a spike signal is included in the signal sections based on a change in the neural signal, and determining whether to transmit the neural signal based on whether the spike signal is included in the signal sections.

### CROSS-REFERENCE TO RELATED APPLICATION

This application claims priority from Korean Patent Application No. 10-2025-0018290, filed on Feb. 12, 2025, in the Korean Intellectual Property Office, the entire disclosure of which is incorporated herein by reference for all purposes.

### BACKGROUND

### 1. Field

Methods and apparatuses consistent with embodiments relate to a method of detecting neural signals, a detection device, and a neural signal measuring system.

### 2. Description of the Related Art

A human or an animal may have trillions or more of nerve cells. Attempts to measure electrical signals emitted by an individual cell or a determined number or more of cell clusters using electrodes are being made to directly measure the status of trillions or more nerve cells. As the size of a detection device is reduced due to miniaturization of electrodes and circuit technology, it has become possible to measure signals at an individual cell level. At the same time, the number of channels for recording activities of as many cells as possible is gradually increasing from tens to thousands. Accordingly, the volume of data measured through multiple channels and the volume of data to be transmitted are also increasing.

### SUMMARY

One or more embodiments may address at least the above problems and/or disadvantages and other disadvantages not described above. Also, the embodiments are not required to overcome the disadvantages described above, and an embodiment may not overcome any of the problems described above.

There is provided a method of detecting a neural signal, the method including: dividing a neural signal, measured via at least one or more of multiple channels, into signal sections each of a specified length; determining, based on detecting a change in the neural signal, whether a spike signal is included in one or more of the signal sections; and selectively controlling transmission of the neural signal based on whether the spike signal is determined to be included in the one or more of the signal sections.

Determining whether the spike signal is included in the one or more of the signal sections may be based on determining whether a size of the change in the neural signal is greater than a threshold.

The size of the change in the neural signal may be determined based on a difference between amplitude sizes of samples of the neural signal spaced apart by a length specified based on a sampling rate of sampling the neural signal.

Determining whether the spike signal is included in the one or more of the signal sections may include identifying, by a neural network, a shape of a waveform included in the one or more of the signal sections.

The neural network may be configured to output, based on the shape of the waveform, at least one of the shape of the waveform and whether to transmit the neural signal.

Determining whether the spike signal is included in the one or more of the signal sections may be based on determining a shape of a waveform of the neural signal.

Selectively controlling transmission of the neural signal may include: setting, based on determining the one or more of the signal sections as comprising the spike signal, the one or more of the signal sections as a target section; and transmitting at least part of the neural signal as included in the target section.

Selectively controlling transmission of the neural signal may include: grouping the spike signal together with a second neural signal into a grouped neural signal, the second neural signal being measured via another channel, of the multiple channels and other than at least one of the one or more of the multiple channels; and transmitting the grouped neural signal.

Grouping the spike signal together with the second neural signal into the grouped neural signal may be based on a correlation analysis result between the spike signal and the second neural signal generated in the another channel.

Dividing of the neural signal into the signal sections each of the specified length may include dividing the signal sections at least partly into overlapping areas comprising the neural signal.

There is provided a detection device including: a communication module configured to receive a neural signal measured via at least one or more of multiple channels; and a processor configured to: divide the neural signal into signal sections each of a specified length; determine, based on detecting a change in the neural signal, whether a spike signal is included in the one or more of the signal sections; and selectively controlling transmission of the neural signal based on whether the spike signal is determined to be included in the one or more of the signal sections.

The processor may be further configured to determine whether the spike signal is included in the one or more signal sections based on determining whether a size of the change in the neural signal is greater than a threshold.

The size of the change in the neural signal may be determined based on a difference between amplitude sizes of samples of the neural signal spaced apart by a length specified based on a sampling rate of sampling the neural signal.

The processor may be further configured to: determine whether the spike signal is included in the one or more of the signal sections based on identifying, by a neural network, a shape of a waveform included in the one or more of the signal sections.

Selectively controlling transmission of the neural signal may include: setting, based on determining the one or more of the signal sections as comprising the spike signal, the one or more of the signal sections as a target section; and transmitting at least part of the neural signal as included in the target section.

Selectively controlling transmission of the neural signal may include: grouping the spike signal together with a second neural signal into a grouped neural signal, the second neural signal being measured via another channel, of the multiple channels and other than at least one of the one or more of the multiple channels; and transmitting the grouped neural signal.

Grouping the spike signal together with the second neural signal into the grouped neural signal may be based on a correlation analysis result between the spike signal and the second neural signal generated in the another channel.

There is provided a neural signal measuring system including: a sensor node configured to convert a neural signal, measured via at least one or more of multiple channels, into a digital signal by an analog-digital converter (ADC), and transmit the digital signal; and a host device configured to store the digital signal transmitted from the sensor node.

The neural signal measuring system may further include: a detection device that may be configured to: divide the neural signal into signal sections each of a specified length; determine, based on detecting a change in the neural signal, whether a spike signal is included in the one or more of the signal sections; and selectively controlling transmission of the neural signal based on whether the spike signal is determined to be included in the one or more of the signal sections, and one of the sensor node and the host device includes the detection device.

The host device may be further configured to: receive, from the detection device, a second neural signal measured via another channel, of the multiple channels and other than at least one of the one or more of the multiple channels; and transmit a result of analyzing a correlation between the spike signal and the second neural signal.

Additional aspects of embodiments will be set forth in part in the description which follows and, in part, will be apparent from the description, or may be learned by practice of the disclosure.

### BRIEF DESCRIPTION OF THE DRAWINGS

The above and/or other aspects will be more apparent by describing certain embodiments with reference to the accompanying drawings, in which:

FIG. 1 is a diagram illustrating an example of a neural signal measured via multiple channels, according to one or more embodiments;

FIG. 2 is a flowchart illustrating a method of detecting a neural signal, according to one or more embodiments;

FIG. 3 is a diagram illustrating a method of determining whether a spike signal is included in signal sections by a shape of a waveform of a neural signal, according to one or more embodiments;

FIG. 4 is a diagram illustrating a method of determining whether a spike signal is included in signal sections, according to one or more embodiments;

FIG. 5A and FIG. 5B are diagrams illustrating a method of determining whether a spike signal is included in signal sections using a neural network, according to one or more embodiments;

FIG. 6 is a diagram illustrating a method of dividing a neural signal into signal sections of a specified length, according to one or more embodiments;

FIG. 7 is a block diagram illustrating a detection device according to one or more embodiments;

FIG. 8 is a flowchart illustrating an operating method of a detection device, according to one or more embodiments; and

FIG. 9A, FIG. 9B, and FIG. 9C are diagrams illustrating configurations of a neural signal measuring system, according to one or more embodiments.

### DETAILED DESCRIPTION

The following detailed structural or functional description is provided as an example only and various alterations and modifications may be made to the embodiments. Thus, an actual form of implementation is not construed as limited to the embodiments described herein and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, and similarly, the second component may also be referred to as the first component.

It should be noted that when one component is described as being “connected,” “coupled,” or “joined” to another component, the first component may be directly connected, coupled, or joined to the second component, or a third component may be “connected,” “coupled,” or “joined” between the first and second components.

The singular forms “a,” “an,” and “the” are intended to include the plural forms as well, unless the context clearly indicates otherwise. It will be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical and scientific terms have the same meanings as those commonly understood by one of ordinary skill in the art to which this disclosure pertains. Terms such as those defined in commonly used dictionaries are to be interpreted as having a meaning that is consistent with their meaning in the context of the relevant art and are not to be interpreted in an idealized or overly formal sense unless expressly so defined herein.

Hereinafter, the embodiments are described in detail with reference to the accompanying drawings. When describing the embodiments with reference to the accompanying drawings, like reference numerals refer to like components and a repeated description related thereto is omitted.

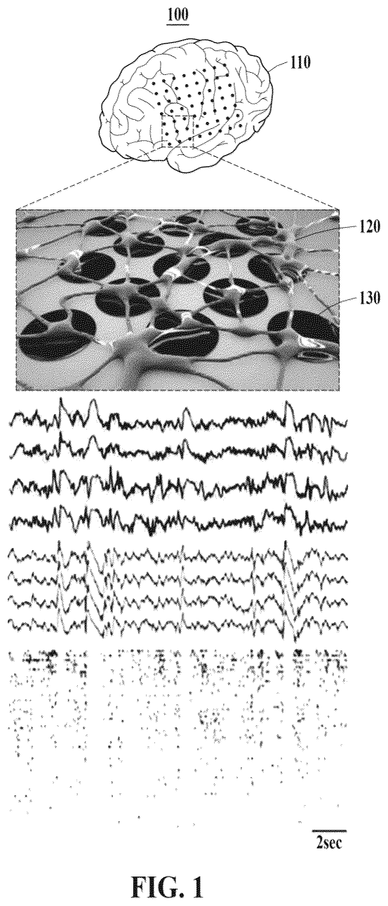

FIG. 1 is a diagram illustrating an example of a neural signal measured via multiple channels, according to one or more embodiments. Referring to FIG. 1, a drawing 100 is illustrated showing neural signals of neurons 120 (or nerve cells) of a brain 110 measured by electrodes 130, according to one or more embodiments.

A variety of methods may be used to analyze a human biometric signal. For example, neural signals of the neurons 120 measured by a plurality of electrodes may be used to analyze brain waves of the brain 110. For ease of description, a nerve cell, or a neuron, is mainly described herein. However, embodiments are not necessarily limited thereto, and the same may be applied to various cell signals of, for example, a muscle cell or a skin cell, in addition to a nerve cell.

A signal transmission method of the brain 110 may be interpreted by flowing a current of a determined size through the nerve cells of the brain 110 and observing intracellular signals. The electrodes 130 may be electrodes for stimulating and sensing cells including nerve cells. The cells may generate at least one of an electrical signal, an optical signal, and/or a chemical signal through a neural stimulation by the electrodes 130.

The electrodes 130 may be, for example, multielectrode array (MEA) electrodes, but are not necessarily limited thereto. As the technology for measuring signals of the neurons 120 using MEA electrodes having multiple channels develops, a number of channels for measuring signals and an amount of data to be transmitted are increasing. Here, the number of channels may correspond to a number of the electrodes 130. The electrodes 130 may also be referred to as “nodes.”

A neural signal may change rapidly within a 1 millisecond(ms) time range and may thus be sampled at a sampling rate of analog-to-digital conversion of 10 kHz or faster. In addition, a neural signal may require a high resolution because a neural signal has a small signal size, for example, in microvolts (μVs). For example, a neural signal may be generated as data of more than or equal to 78 megabytes (Mbytes) per second. When recalculating this, 4.7 gigabytes (GBytes) of data may be generated per minute. When a number of channels measuring neural signals increases to thousands, a large amount of data may continuously be generated, which may cause a bottleneck in data processing. When multiple channels are used to measure neural signals as described above, an amount of data transmission and/or storage may be significantly increased.

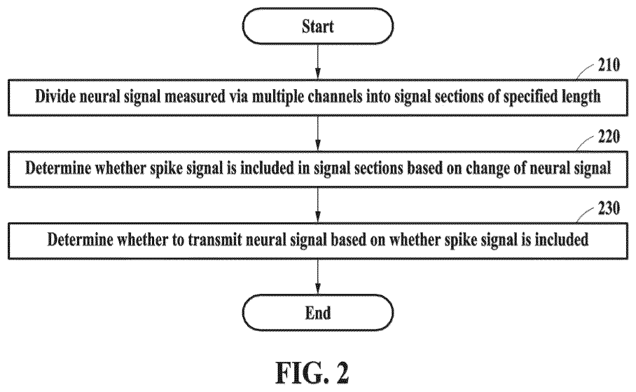

FIG. 2 is a flowchart illustrating a method of detecting a neural signal, according to one or more embodiments. Operations of the embodiments to be described with reference to FIG. 2 and below may be performed sequentially but not necessarily. For example, the order of the operations may change, and at least two of the operations may be performed in parallel or one operation may be performed separately.

Referring to FIG. 2, a detection device may transmit a neural signal depending on whether to transit the neural signal, which may be determined through operations, such as operation 210, operation 220, and operation 230. The detection device may be, for example, a detection device 700 illustrated in FIG. 7, but is not necessarily limited thereto.

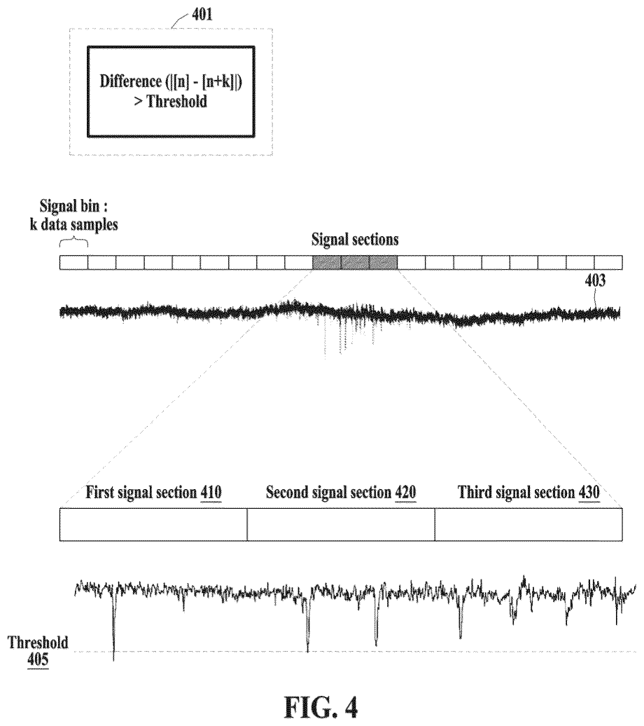

In operation 210, the detection device may divide the neural signal measured via multiple channels into signal sections of a specified length. Here, a signal section may correspond to one signal bin including, for example, k (k is a natural number>1) samples of neural signals as illustrated in FIG. 4. The signal bin may indicate, for example, a specific frequency component of a signal in a frequency domain.

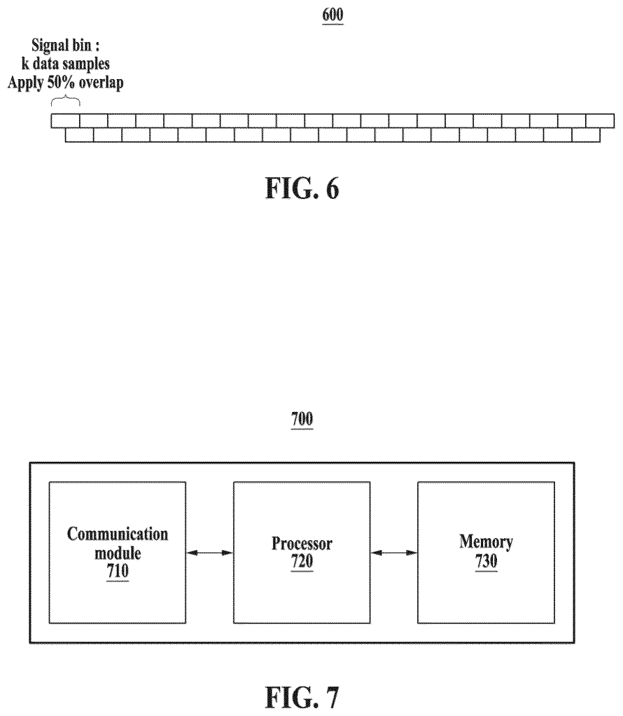

Alternatively, the detection device may divide the signal sections to have overlapping areas so that sections including the neural signal may overlap one another. The method, performed by the detection device, of dividing the signal sections so that the neural signal overlaps is described in detail below with reference to FIG. 6.

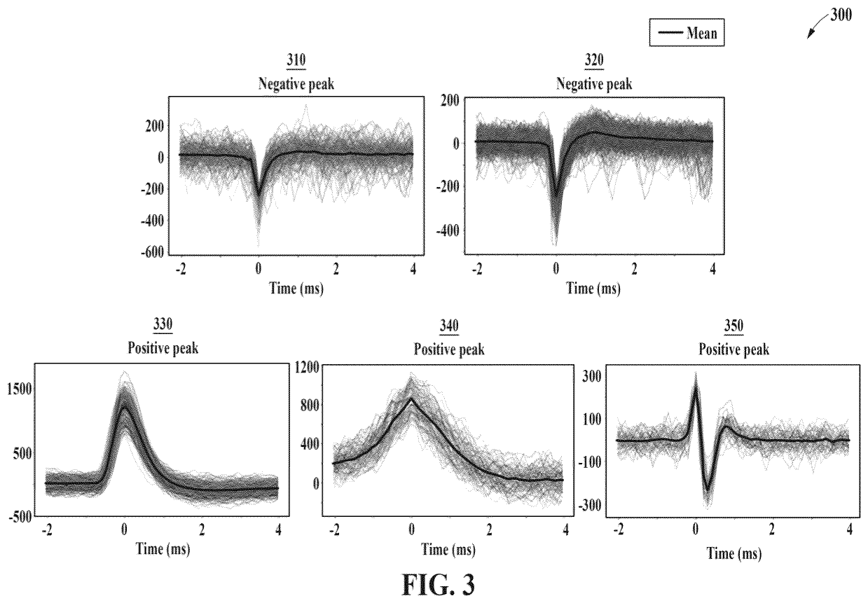

In operation 220, the detection device may determine whether a spike signal is included in the signal sections divided in operation 210 based on a change in the neural signal. The detection device may determine whether a spike signal is included in the signal sections based on a shape of a waveform of the neural signal. The method, performed by the detection device, of determining whether a spike signal is included in the signal sections by the shape of the waveform of the neural signal is described in detail below with reference to FIG. 3.

The detection device may determine whether a spike signal is included in the signal sections based on whether a size of the change in the neural signal is greater than a threshold. The size of the change in the neural signal may be determined based on a difference between amplitude sizes ((Difference (|[n]−[n+k]|) or a difference between one sample neural signal or signal section (n) compared to another sample neural signal or signal section (n+k) as shown with block 401) of samples of the neural signal spaced apart by a length specified by considering a sampling rate of the neural signal. The threshold may be a value corresponding to a change in an amplitude of a spike signal, but is not necessarily limited thereto. A method, performed by the detection device, of determining whether a spike signal is included in signal sections based on whether the size of the change in the neural signal is greater than the threshold is described in detail below with reference to FIG. 4.

The detection device may determine whether a spike signal is included in the signal sections by further considering a shape of a waveform, identified by a neural network, of a signal section including a neural signal with the size of the change in the neural signal greater than the threshold. The neural network may output at least one of the shape of the waveform of the neural signal or whether to transmit the neural signal according to the shape of the waveform of the neural signal. Here, training of a neural network may be performed differently depending on the neuron, or the nerve cell, to be measured. The detection device may train and use different neural networks according to a kind or type of waveforms of neural signals.

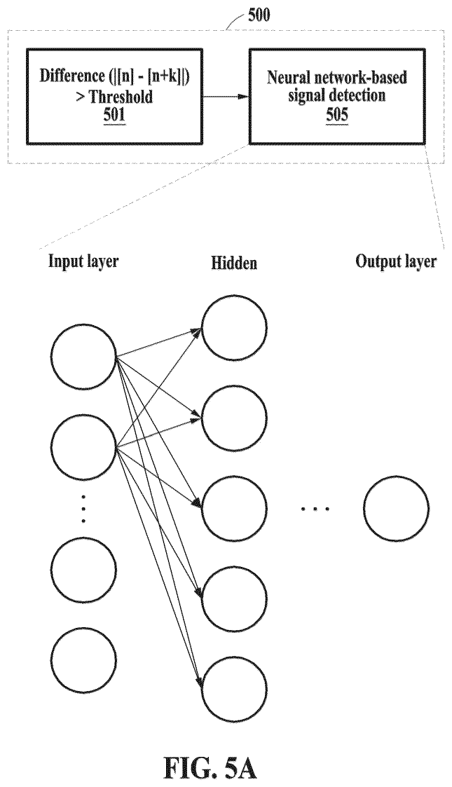

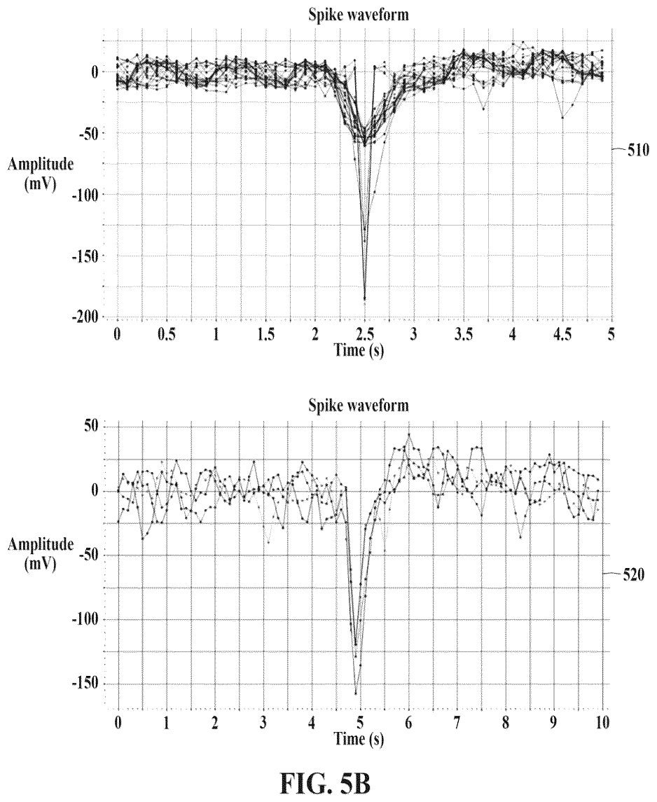

A method, performed by the detection device, of determining whether a spike signal is included in the signal sections by further considering the shape of the waveform, identified by the neural network, of the signal section is described in detail below with reference to FIG. 5A and FIG. 5B.

In addition, when determining a signal section including a spike signal, the detection device may determine that not only a target channel, in which a neural signal corresponding to the spike signal is measured, but also another channel related to the target channel include a spike signal. Here, “another channel related to the target channel” may indicate another channel that has a relationship with the target channel, such as, for example, a channel that is positionally adjacent to the target channel, a channel that has a same type of neural signal to be measured as the target channel, a channel in which a neural signal is detected at the same time as the target channel, a channel in which a neural signal showing a similar shape of waveform to the target channel is detected at a certain time interval from the target channel, and/or a channel showing an identical or similar shape of waveform to the shape of the waveform shown in the target channel. For example, when it is determined that a signal section corresponding to channel a includes a spike signal, the detection device may determine that a spike signal is also included in a signal section corresponding to channel b, which is specified at the same time as a, or channel c, which is adjacent to channel a.

In operation 230, the detection device may determine whether to transmit a neural signal based on whether a spike signal is included, which is determined in operation 220. When the signal sections include the spike signal, the detection device may determine a signal section including the spike signal as a target section. The detection device may transmit a neural signal included in the target section.

According to one or more embodiments, when the signal sections include the spike signal, the detection device may group the spike signal together with a neural signal generated in another channel related to a target channel corresponding to the target section including the spike signal. The detection device may transmit the grouped neural signals.

For example, the detection device may group the spike signal together with a neural signal corresponding to a signal section of channels having a shape of a waveform in a negative peak shape, such as in graph 310 and graph 320 described below with reference to FIG. 3, or may group the spike signal together with a neural signal corresponding to a signal section of channels having a shape of a waveform in a positive peak shape, such as in graph 330 and graph 340. Here, whether the shape of the waveform shows a negative peak shape or a positive peak shape may vary depending on a connection relationship between electrode(s) and cell(s). Here, the “connection relationship” between electrode(s) and cell(s) may be considered as a temporal connection relationship between cell signals. For example, after a signal having a positive peak shape occurs in cell A, a signal having a positive peak or negative peak shape may occur in cell B simultaneously or within a certain period of time (e.g., several ms or tens of ms). In a case in which signals having a positive peak shape and a negative peak shape are repeatedly observed with a certain probability, the relationship between the two cells may be considered as the connection relationship.

The detection device may also group the spike signal together with a neural signal corresponding to a signal section of channels having an identical shape of waveforms.

Alternatively, the detection device may group the spike signal together with a neural signal corresponding to a signal section of channels having a shape of a waveform in a mixed spike shape, which changes from a positive peak into a negative peak as in a graph 350 below, or which changes from a negative peak into a positive peak. In a case of a neural signal that shows a mixed spike shape, the shape of the waveform may vary depending on a position of the nerve cell being measured. The detection device may set an importance of the neural signal showing a mixed spike shape to be lower than an importance of a neural signal showing a positive peak shape or a negative peak shape. When determining whether to transmit a neural signal, the detection device may consider the importance of each neural signal too to finally determine whether to transmit the neural signal.

The detection device may group the spike signal with the neural signal generated in the another channel based on a correlation analysis result between the spike signal and the neural signal generated in the another channel. Here, the “correlation” between the spike signal and the neural signal generated in the another channel may be interpreted to refer to not only a mathematically defined formula, but also a wide range of correlations that include similarity, correlation, and cross correlation between the spike signal and the neural signal generated in the another channel.

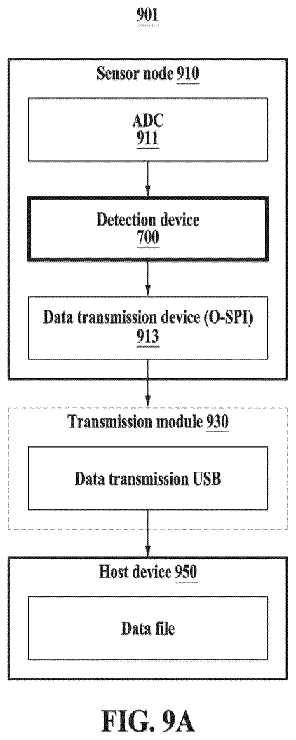

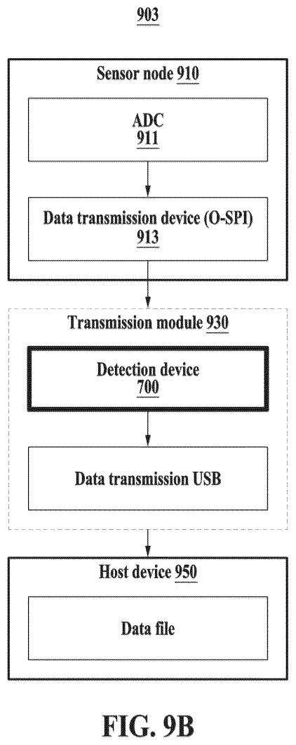

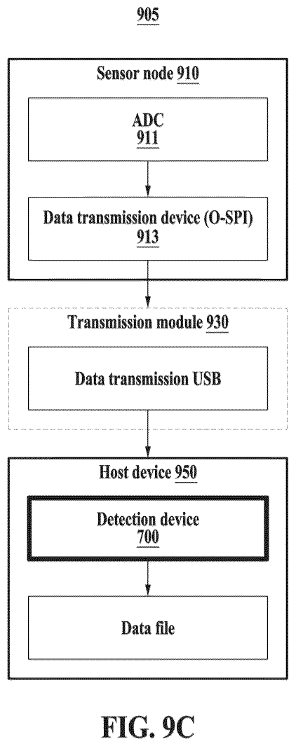

Here, the correlation analysis results may be received from a host device 950 of neural signal measuring systems, such as neural signal measuring system 901, neural signal measuring system 903, and neural signal measuring system 905, described below with reference to FIG. 9A, FIG. 9B, and FIG. 9C or may be directly analyzed by the detection device.

FIG. 3 is a diagram illustrating a method of determining whether a spike signal is included in signal sections by a shape of a waveform of a neural signal, according to one or more embodiments. Referring to FIG. 3, graphs, such as graph 310, graph 320, graph 330, graph 340, and graph 350, are illustrated showing waveforms of various types of neural signals, according to one or more embodiments.

A “spike signal” may refer to a signal having waveforms of a neural signal that show a rapid change in size over time, as in the graphs, such as graph 310, graph 320, graph 330, graph 340, and graph 350,. The graph 310 and graph 320 illustrate a negative peak shape, which is a spike having a shape of a waveform that goes below a baseline. The graph 330 and graph 340 illustrate a positive peak shape, which is a spike having a shape of a waveform that rises above the baseline. The graph 350 illustrates a mixed spike shape, in which the shape of a waveform changes from a positive peak to a negative peak.

According to one or more embodiments, the amount of data transmission and storage may be reduced by selectively transmitting and/or storing a neural signal that has a characteristic of changing rapidly over time in a certain signal section (i.e., a “spike signal”), as illustrated in the graphs, such as graph 310, graph 320, graph 330, graph 340, and graph 350,.

FIG. 4 is a diagram illustrating a method of determining whether a spike signal is included in signal sections, according to one or more embodiments. Referring to FIG. 4, shapes of waveforms of one or more of neural signals 403 in signal sections (e.g., a first signal section 410, a second signal section 420, and a third signal section 430) are illustrated.

Since an amplitude of each neural signal 403 in the signal sections, such as first signal section 410, second signal section 420, and third signal section 430, illustrated in FIG. 4 occurs uniformly within a certain range, a length between samples of the neural signal 403 may tend to have a certain amount of variation.

The detection device may first determine a signal section to be transmitted by determining, based on a size of a change in the one or more of neural signals 403 measured in the signal sections, such as first signal section 410, second signal section 420, and third signal section 430, divided into a specified length, whether a corresponding neural signal corresponds to a spike signal to be transmitted. Here, the size of the change in at least one of the neural signals 403 may be determined based on a difference between signal sizes of samples spaced apart by a length specified by considering a sampling rate of each neural signal 403.

The detection device may determine, based on the size of the change in the neural signals 403 measured in the signal sections, such as first signal section 410, second signal section 420, and third signal section 430, whether a spike signal having the size of the change in a neural signal greater than a threshold 405, such as according to block 401, is included in the signal sections, such as first signal section 410, second signal section 420, and third signal section 430. For example, the size of the change in a neural signal included in the first signal section 410 and the second signal section 420 may be greater than the threshold 405, and the size of the change in a neural signal included in the third signal section 430 may be less than the threshold 405.

In this case, the detection device may determine that a spike signal is included in a signal section including at least one of the neural signals 403 having the size of the change greater than the threshold 405, that is, the first signal section 410 and the second signal section 420. The detection device may transmit the at least one or more of the neural signals 403 included in the first signal section 410 and the second signal section 420 respectively and may not transmit a neural signal included in the third signal section 430.

FIG. 5A and FIG. 5B are diagrams illustrating a method of determining whether a spike signal is included in signal sections using a neural network, according to one or more embodiments.

Referring to FIG. 5A, a drawing 500 is illustrated describing a method, performed by a detection device, of determining whether a spike signal is included in signal sections, by further using a neural network secondarily.

As described above, the detection device may first determine (or select) a signal section to be transmitted by determining whether a spike signal, which has a size of a change in the neural signal greater than a threshold, such as at block 501, is included in the signal sections. Here, a process of determining the signal section first may be performed before or immediately after an analog-to-digital conversion (ADC) process of a sensor node 910 in a neural signal measuring system described below with reference to FIG. 9A, FIG. 9B, and FIG. 9C.

The detection device may finally determine whether a spike signal is included in the signal sections by secondarily considering a shape of a waveform, identified by a neural network, of the signal section including the neural signal with the size of the change greater than the threshold, such as at block 505, regarding the signal section that is first determined (e.g., the signal section including the neural signal with the size of the change greater than the threshold).

The neural network may determine whether a spike signal is included in the corresponding signal section based on whether a shape of a waveform of a signal section including a neural signal, which has a size of a change of the detected neural signal greater than the threshold, is similar to the shape of the waveform of the neural signal detected in the signal section that is first determined as described above. Here, the neural network may output at least one of the shape of the waveform of the neural signal or whether to transmit the neural signal according to the shape of the waveform of the neural signal.

The neural network may include an input layer, a hidden layer, and an output layer. There may be a same number of input layers as the number of length units of the neural signal, that is, the number of signal sections. The output layer may output one or more values. The neural network may output a value that simply determines whether to transmit the neural signal or may output a type (or kind) of the waveform detected in a target signal section. A weight value of the neural network may be stored in memory (e.g., a memory 730 of FIG. 7) of the detection device. A model structure of the neural network may be fixed hardware or may be changed through external settings.

The detection device may prepare and use multiple neural networks for each waveform of the neural signal. The detection device may train and use different neural networks according to conditions for measuring neural signals or the type of waveform to be detected.

The detection device may improve the accuracy in selecting a neural signal to transmit by determining whether to transmit the neural signal, considering not only a change in an amplitude of the neural signal but also a shape of the neural signal (or a shape of the signal section) through the neural network.

Referring to FIG. 5B, a graph 510 is illustrated showing a waveform of a neural signal included in a signal section determined first before applying a neural network, and a graph 520 is illustrated showing a waveform of a neural signal detected after applying the neural network.

The waveform shown in the graph 510 may include a signal corresponding to noise of the detection device itself. In this case, the neural network may obtain a more accurate waveform of the neural signal as in the graph 520 by removing noise from the neural signal, such as one shown in the graph 510, which is included in the signal section that is determined first.

FIG. 6 is a diagram illustrating a method of dividing a neural signal into signal sections of a specified length, according to one or more embodiments. Referring to FIG. 6, a drawing 600 is illustrated showing the signal sections that are divided to have overlapping areas, according to one or more embodiments.

When dividing a measured neural signal into signal sections of a specified length, the detection device may divide the signal sections to have overlapping areas so that sections including the neural signal may overlap one another, as shown in the drawing 600. The detection device may minimize missing data (e.g., the neural signal) at a boundary point of each signal section by setting the signal sections to be overlapping sections in which the neural signal overlaps by, for example, 50%.

FIG. 7 is a block diagram illustrating a detection device according to one or more embodiments. Referring to FIG. 7, the detection device 700 may include a communication module 710 and a processor 720. The detection device may further include the memory 730)

The communication module 710 may receive a neural signal measured via multiple channels. The communication module 710 may include a Wi-Fi and Bluetooth that may perform data communication between the detection device 700 and an external device and/or a fifth-generation (5G) module that supports a real-time control and a remote monitoring through high-speed data transmission.

The processor 720 may divide the neural signal received via the communication module 710 into signal sections of a specified length. The processor 720 may determine whether a spike signal is included in the signal sections based on a change in the neural signal. The processor 720 may determine whether to transmit the neural signal based on whether a spike signal is included in the signal sections.

The processor 720 may be one or more and may execute instructions or programs or control the detection device 700. The processor 720 may include, for example, a graphics processing unit (GPU), a neural processing unit (NPU), a tensor processing unit (TPU), and the like. In addition, in some embodiments, the processor 720 may include a central processing unit (CPU).

The processor 720 may perform the operations described above with reference to FIG. 1, FIG. 2, FIG. 3, FIG. 4, FIG. 5A, FIG. 5B, and FIG. 6 as at least some of the instructions stored in the memory 730 are executed by one or more processors 720.

The memory 730 may store a neural network and/or a weight value of the neural network. The memory 730 may be electrically connected to the processor 720 and may store instructions executed by the processor 720. The memory 730 may store instructions executable by the processor 720. The memory 730 may be volatile memory or non-volatile memory.

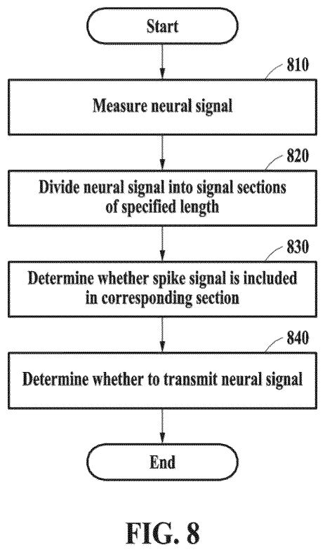

FIG. 8 is a flowchart illustrating an operating method of a detection device, according to one or more embodiments. Referring to FIG. 8, a detection device may determine whether to transmit a neural signal through operation 810, operation 820, operation 830, and operation 840.

In operation 810, the detection device may measure a neural signal. The detection device may provide neural stimulation to nerve cells, for example, by a plurality of multielectrode arrays (MEAs) including electrodes for stimulating and sensing cells. The nerve cells may generate at least one of an electrical signal, an optical signal, and/or a chemical signal through a neural stimulation. The detection device may measure a signal generated by the nerve cells (i.e., a “neural signal”).

In operation 820, the detection device may divide the neural signal measured in operation 810 into signal sections of a specified length.

In operation 830, the detection device may determine whether there is a spike signal, which has a rapid change in an amplitude size of the neural signal, within the signal sections divided in operation 820.

In operation 840, the detection device may determine whether to transmit a neural signal based on a result of the determination made in operation 830. The detection device may determine not to transmit a neural signal in a signal section that includes signals of which the size of the change in the neural signal is not large. The detection device may determine to transmit a neural signal in a signal section that includes a signal (e.g., a spike signal) that has a rapid change in an amplitude size of the neural signal.

FIG. 9A, FIG. 9B, and FIG. 9C are diagrams illustrating configurations of a neural signal measuring system, according to embodiments. Referring to FIG. 9A, FIG. 9B, and FIG. 9C, diagrams are illustrated showing the configurations of neural signal measuring systems, such as neural signal measuring system 901, neural signal measuring system 903, and neural signal measuring system 905, according to one or more embodiments.

The neural signal measuring systems, such as neural signal measuring system 901, neural signal measuring system 903, and neural signal measuring system 905, may each include the sensor node 910 and the host device 950. The neural signal measuring systems, such as neural signal measuring system 901, neural signal measuring system 903, and neural signal measuring system 905, may further include a transmission module 930. The neural signal measuring systems, such as neural signal measuring system 901, neural signal measuring system 903, and neural signal measuring system 905, may further include the detection device 700.

The sensor node 910 may convert a neural signal measured via multiple channels into a digital signal by using an analog-to-digital converter (ADC) 911 and transmit the digital signal. The sensor node 910 may include the electrodes described above, the ADC 911, and/or a data transmission device 913.

The data transmission device 913 may transmit data using, for example, the O-SPI protocol, which is a form of serial peripheral interface (SPI). The SPI protocol may be a serial communication protocol for data transmission between a microcontroller and a peripheral device. The SPI protocol may use a master-slave structure, have a high data transmission speed, and support full duplex communication. The SPI protocol is mainly used for communication with various peripheral devices such as a sensor, memory, and/or a display and may have a high data transmission speed and a simple hardware structure.

The transmission module 930 may be a data transmission interface such as, for example, a universal serial bus (USB). The transmission module 930 may be included in, for example, a head-mounted display (HMD). However, embodiments are not necessarily limited thereto. The transmission module 930 may be optionally included in the neural signal measuring systems, such as neural signal measuring system 901, neural signal measuring system 903, and neural signal measuring system 905.

The host device 950 may store a signal (e.g., a digital signal) transmitted from the sensor node 910.

The host device 950 may receive, from the detection device 700, a neural signal generated in another channel related to a channel corresponding to a target section in which a spike signal is generated. The host device 950 may analyze a correlation between the spike signal and the neural signal generated in the another channel and may transmit the analysis result to the detection device 700.

Alternatively, the host device 950 may receive, from the detection device 700, neural signals generated in signal sections of all channels corresponding to a first few times (e.g., 1 to 3 times) to perform analysis on a relationship between multiple channels and may group a target channel with another channel related to the target channel as one group based on a result of the analysis. The host device 950 may transmit information on the grouped group(s) to the detection device 700, thereby causing the detection device 700 to transmit neural signals of the grouped group(s) to the host device 950.

The host device 950 may include, for example, various computing devices such as a mobile phone, a smartphone, a tablet personal computer (PC), an electronic book (e-book) device, a laptop, a PC, a desktop, a workstation, or a server, various wearable devices such as a smart watch, smart eyeglasses, a head-mounted display (HMD), or smart clothing, various home appliances such as a smart speaker, a smart television (TV), or a smart refrigerator, and other devices such as a smart car, a smart kiosk, an Internet of Things (IoT) device, a walking assist device (WAD), a drone, or a robot.

The detection device 700 may divide the neural signal into signal sections of a specified length. The detection device 700 may determine whether a spike signal is included in the signal sections based on a change in the neural signal. The detection device 700 may determine whether to transmit a neural signal based on whether the spike signal is included.

The detection device 700 may be included in the sensor node 910 as illustrated in the neural signal measuring system 901 of FIG. 9A, in the transmission module 930 as illustrated in the neural signal measuring system 903 of FIG. 9B, or in the host device 950 as illustrated in the neural signal measuring system 905 of FIG. 9C, considering at least one of data transmission efficiency or storage capacity.

For example, when the detection device 700 is included in the sensor node 910 as in FIG. 9A, the detection device 700 may determine whether data of a neural signal converted into a digital signal by the ADC 911 includes the spike signal to determine whether to transmit the corresponding data.

When the detection device 700 determines whether to transmit the data, control of the data transmission device 913 may be performed through a separate circuit or by a protocol used to transmit the data.

Whether a significant signal is included in a neural signal to be transmitted and whether to transmit the neural signal may be determined by the sensor node 910 as in FIG. 9A, by the transmission module 930 as in FIG. 9B, or by the host device 950 as in FIG. 9C before the data is stored so that the data transmission efficiency and/or storage efficiency may be improved.

When the detection device 700 is included in the sensor node 910 as illustrated in FIG. 9A, an amount of data transmission and storage may be reduced. In addition, when the detection device 700 is included in the host device 950 as in FIG. 9C, the amount of data transmission may increase.

The embodiments described herein may be implemented using a hardware component, a software component, and/or a combination thereof. For example, a processing device may be implemented using one or more general-purpose or special-purpose computers, such as, for example, a processor, a controller, an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing device may also access, store, manipulate, process, and create data in response to execution of the software. For purpose of simplicity, the processing device is described as singular. However, one of ordinary skill in the art will appreciate that a processing device may include multiple processing elements and/or multiple types of processing elements. For example, the processing device may include a plurality of processors, or a single processor and a single controller. In addition, a different processing configuration is possible, such as one including parallel processors.

The software may include a computer program, a piece of code, instructions, or some combination thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software may also be distributed over network-coupled computer systems so that the software is stored and executed in a distributed fashion. The software and data may be stored in a non-transitory computer-readable recording medium.

The methods according to the embodiments may be recorded in non-transitory computer-readable media including program instructions to implement various operations of the embodiments. The media may also include the program instructions, data files, data structures, and the like alone or in combination. The program instructions recorded on the media may be those specially designed and constructed for the embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specially configured to store and perform program instructions, such as ROM, random-access memory (RAM), flash memory, and the like. Examples of program instructions include both machine code, such as those produced by a compiler, and files containing high-level code that may be executed by the computer using an interpreter. The above-described hardware devices may be configured to act as one or more software modules in order to perform the operations of the above-described embodiments, or vice versa.

Although the embodiments have been described with reference to the limited number of drawings, one of ordinary skill in the art may apply various technical modifications and variations based thereon. For example, suitable results may be achieved if the described techniques are performed in a different order and/or if components in a described system, architecture, device, or circuit are combined in a different manner and/or replaced or substituted by other components or their equivalents. Therefore, other implementations, other embodiments, and equivalents to the claims are also within the scope of the following claims.

What is claimed is:

1. A method of detecting a neural signal, the method comprising:
dividing a neural signal, measured via at least one or more of multiple channels, into signal sections each of a specified length;
determining, based on detecting a change in the neural signal, whether a spike signal is included in one or more of the signal sections; and
selectively controlling transmission of the neural signal based on whether the spike signal is determined to be included in the one or more of the signal sections.

2. The method of claim 1, wherein determining whether the spike signal is included in the one or more of the signal sections is based on determining whether a size of the change in the neural signal is greater than a threshold.

3. The method of claim 2, wherein the size of the change in the neural signal is determined based on a difference between amplitude sizes of samples of the neural signal spaced apart by a length specified based on a sampling rate of sampling the neural signal.

4. The method of claim 2, wherein determining whether the spike signal is included in the one or more of the signal sections further comprises identifying, by a neural network, a shape of a waveform included in the one or more of the signal sections.

5. The method of claim 4, wherein the neural network is configured to output, based on the shape of the waveform, at least one of the shape of the waveform and whether to transmit the neural signal.

6. The method of claim 1, wherein determining whether the spike signal is included in the one or more of the signal sections is based on determining a shape of a waveform of the neural signal.

7. The method of claim 1, wherein selectively controlling transmission of the neural signal comprises:
setting, based on determining the one or more of the signal sections as comprising the spike signal, the one or more of the signal sections as a target section; and
transmitting at least part of the neural signal as included in the target section.

8. The method of claim 1, wherein selectively controlling transmission of the neural signal comprises:
grouping the spike signal together with a second neural signal into a grouped neural signal, the second neural signal being measured via another channel, of the multiple channels and other than at least one of the one or more of the multiple channels; and
transmitting the grouped neural signal.

9. The method of claim 8, wherein grouping the spike signal together with the second neural signal into the grouped neural signal is based on a correlation analysis result between the spike signal and the second neural signal generated in the another channel.

10. The method of claim 1, wherein dividing of the neural signal into the signal sections each of the specified length comprises dividing the signal sections at least partly into overlapping areas comprising the neural signal.

11. A detection device comprising:
a communication module configured to receive a neural signal measured via at least one or more of multiple channels; and
a processor configured to:
divide the neural signal into signal sections each of a specified length;
determine, based on detecting a change in the neural signal, whether a spike signal is included in the one or more of the signal sections; and
selectively controlling transmission of the neural signal based on whether the spike signal is determined to be included in the one or more of the signal sections.

12. The detection device of claim 11, wherein the processor is further configured t determine whether the spike signal is included in the one or more signal sections based on determining whether a size of the change in the neural signal is greater than a threshold.

13. The detection device of claim 12, wherein the size of the change in the neural signal is determined based on a difference between amplitude sizes of samples of the neural signal spaced apart by a length specified based on a sampling rate of sampling the neural signal.

14. The detection device of claim 12, wherein the processor is further configured to:
determine whether the spike signal is included in the one or more of the signal sections based on identifying, by a neural network, a shape of a waveform included in the one or more of the signal sections.

15. The detection device of claim 11, wherein selectively controlling transmission of the neural signal comprises:
setting, based on determining the one or more of the signal sections as comprising the spike signal, the one or more of the signal sections as a target section; and
transmitting at least part of the neural signal as included in the target section.

16. The detection device of claim 11, wherein selectively controlling transmission of the neural signal comprises:
grouping the spike signal together with a second neural signal into a grouped neural signal, the second neural signal being measured via another channel, of the multiple channels and other than at least one of the one or more of the multiple channels; and
transmitting the grouped neural signal.

17. The detection device of claim 16, wherein grouping the spike signal together with the second neural signal into the grouped neural signal is based on a correlation analysis result between the spike signal and the second neural signal generated in the another channel.

18. A neural signal measuring system comprising:
a sensor node configured to:
convert a neural signal, measured via at least one or more of multiple channels into a digital signal by an analog-digital converter (ADC); and
transmit the digital signal; and
a host device configured to store the digital signal transmitted from the sensor node.

19. The neural signal measuring system of claim 18, further comprising:
a detection device configured to:
divide the neural signal into signal sections each of a specified length;
determine, based on detecting a change in the neural signal, whether a spike signal is included in the one or more of the signal sections; and
selectively controlling transmission of the neural signal based on whether the spike signal is determined to be included in the one or more of the signal sections,
wherein one of the sensor node and the host device comprises the detection device.

20. The neural signal measuring system of claim 19, wherein the host device is further configured to:
receive, from the detection device, a second neural signal measured via another channel, of the multiple channels and other than at least one of the one or more of the multiple channels; and
transmit a result of analyzing a correlation between the spike signal and the second neural signal.
